(19) United States
(12) Patent Application Publication
(10) Pub. No.: US 2021/0361565 A1
(43) Pub. Date: Nov. 25, 2021

(54) Oral Pharmaceutical Composition of an NK-1 Antagonist

(71) Applicant: Intervet Inc., Madison, NJ (US)

(72) Inventor: Chen-Chao Wang, West Windsor, NJ (US)

(73) Assignee: Intervet Inc., Madison, NJ (US)

(21) Appl. No.: 16/765,883

(22) PCT Filed: Dec. 20, 2018
(86) PCT No.: PCT/EP2018/086078

§ 371 Date: May 21, 2020

Related U.S. Application Data
(60) Provisional application No. 62/608,636, filed on Dec. 21, 2017.

(51) Int. Cl. A61K 9/00; A61K 31/437; A61K 47/18; A61K 47/26; A61K 47/46

(52) U.S. Cl. CPC A61K 9/0053; A61K 31/437; A61K 47/46; A61K 47/26; A61K 47/18

(57) ABSTRACT

An oral pharmaceutical composition comprising telmapitant, a non-aqueous solvent and one or more additional pharmaceutical acceptable excipients wherein the telmapitant is in solution in the composition. A method of treatment or prevention of emesis in animals comprising administering the oral pharmaceutical composition.

### BACKGROUND

U.S. Pat. No. 7,049,320 discloses compounds of Formula I which are an NK₁ antagonists and useful in the treatment of delayed onset emesis such as experienced one to several days after receiving chemotherapy. U.S. Pat. No. 7,049,320 discloses that compounds of Formula I can be in liquid form preparations including solutions, suspensions and emulsions. Oral administration is also disclosed.

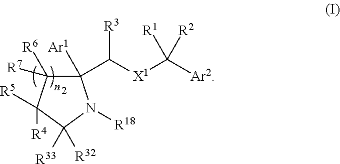

The compound rolapitant (Varubi™), a compound disclosed in U.S. Pat. No. 7,049,320, is approved for use in humans. It has been shown to be efficacious and safe for the prevention of chemotherapy induced nausea and vomiting (Rapoport et al, European Journal of Cancer, 57 (2016) pp 26-30).

The compound telmapitant or (5R,8S)-8-[[(1R)-1-[3,5-bis(trifluoromethyl)phenyl]ethoxy]methyl]-8-phenyl-1,3,9-triazaspiro[4.5]decane-2,4-dione, CAS #552292-58-7, is also disclosed in U.S. Pat. No. 7,049,320.

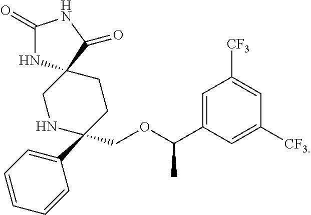 [SMILES: C[C@@H](OC[C@@]1(c2ccccc2)CC[C@]2(CN1)NC(=O)NC2=O)c1cc(C(F)(F)F)cc(C(F)(F)F)c1]

The tachykinin NK-1 receptor is part of a family of receptors that also includes the NK-2 and NK-3 receptors (L Quartara and C A Maggi, 1997, The tachykinin NK₁ receptor. Part I: ligands and mechanisms of cellular activation. Neuropeptides 31(6), 537-563).

The natural and most potent agonist for the NK-1 receptor is the tachykinin substance P. In the CNS, NK-1 receptors have been shown to be involved in behavioral responses, regulation of cardiovascular and respiratory function, and activating the emetic reflex. NK-1 antagonists have proven to be very effective antiemetics with distinct advantages over other classes of antiemetics. NK-1 antagonists have achieved regulatory approval for an antiemetic indication in both humans (aprepitant, i.e. Emend® and rolapitant, i.e. Varubi®), and in dogs (maropitant, i.e. Cerenie®). In dogs, maropitant had been shown to be effective against both centrally acting emetogens (apomorphine IV) and peripherally acting emetogens (syrup of ipecac orally). (See H S Sedlecek, et. al. 2008, J. Vet. Pharmacol. Therap. 31(6) 533-537).

NK-1 antagonist are also effective in treating postsurgical/post anesthesia-induced emesis, motion induced emesis, and emesis from disease (D S Ramsey, et. al. 2008, Safety and efficacy of injectable and oral maropitant, a selective neurokinin₁ receptor antagonist, in a randomized clinical trial or treatment of vomiting in dogs. J. Vet. Pharmacol. Therap. 31(6) 538-543).

Cerenia® (maropitant citrate) tablets are approved for the prevention of acute vomiting and vomiting due to motion sickness (See NADA 141-262, approved Jan. 29, 2007). Cerenia® is also available as an injectable solution approved for the treatment of acute vomiting (See NADA 141-263, approved Jan. 29, 2007).

None of the above reference discloses the inventive non-aqueous telmapitant solution formulations.

### SUMMARY OF THE INVENTION

An oral pharmaceutical composition comprising telmapitant, a non-aqueous solvent and one or more additional pharmaceutical acceptable excipients wherein the telmapitant is in solution in the composition.

A method of treating or preventing emesis in an animal comprising administering orally to the animal an effective dose of the above oral pharmaceutical composition.

### DESCRIPTION OF THE FIGURES

FIG. 1—comparison of long term PK profiles for the comparative example formulations (injectable, tablet and suspension) and the solution formulations of Example 1.

FIG. 2—comparison of short term PK profiles for the comparative example formulations (injectable, tablet and suspension) and the solution formulations of Example 1.

FIGS. 3A-3E—Inter-individual variability of the PK profiles of the solution formulations (FIGS. 3A-3C) and the comparative example formulations (injectable, tablet and suspension) (FIGS. 3D and 3E) of Example 1.

FIGS. 4A-4B—Differences in onset of antiemetic effects between liquid and solid telmapitant formulations. Number of vomits (FIG. 4A) and number of retches (FIG. 4B).

FIGS. 5A-5B—Number of vomits (FIG. 5A) and retches (FIG. 5B) in an apomorphine-induced emesis model in dogs: Cerenia® (maropitant) administered in daily doses of 2 mg/kg and telmapitant administered in single doses of 5 and 7 mg/kg.

### DETAILED DESCRIPTION

### Definitions

d-α-Tocopherol polyethylene glycol 1000 succinate, CAS #9002-96-4 (TPGS) or vitamin E TPGS is a water-soluble derivative of the natural form of vitamin E, d-α-tocopherol. It is produced by the esterification of crystalline d-α-tocopheryl succinate by polyethylene glycol 1000. It is a solubilizer of poorly soluble drugs and an absorption enhancer.

Phosal® 50 PG is standardized phosphatidylcholine concentrate (PC) with at least 50% PC in propylene glycol (See FDA drug master file No. 13931).

Propylene glycol, CAS #57-55-6, is propane-1,2-diol, an organic compound that is miscible in a wide range of solvents.

Oleic acid, CAS #112-80-1 is a omega 9 fatty acid with a formula of CH₃(CH₂)₇CH═CH(CH₂)₇COOH.

Ethylenediaminetetraacetic acid tetrasodium salt dihydrate, CAS #10378-23-1 (EDTA 4Na) is used for medical and industry purposes including as an antioxidant.

Sucralose or 1,6-Dichloro-1,6-dideoxy-β-D-fructofuranosyl-4-chloro-4-deoxy-α-D-galactopyranoside, CAS #56038-13-2, is an artificial sweetener and sugar substitute.

Soluplus is a polyvinyl caprolactam-polyvinyl acetate-polyethylene glycol graft copolymer is an excipient used to improve solubility and bioavailability for poorly soluble active ingredients.

Transcutol HP—highly purified diethylene glycol monoethyl ether NF, a multifunctional solvent.

Medium-chain triglycerides (MCTs) are triglycerides whose fatty acids have an aliphatic tail of 6-12 carbon atoms.

Miglyol® 810 and Miglyol®812N are triglycerides of fractionated plant fatty acids of C₈ and C₁₀. They are triglycerides of coconut oil. They are caprylic capric triglyceride. MIGLYOL® 810/812 differs only in their C₈/C₁₀-ratios. Due to its low C10-content, the viscosity and cloud point of MIGLYOL® 810 is lower.

Non-aqueous solvent is an organic solvent or a liquid lipid material.

Anti-emetic effect means control of vomiting. Antiemetic drugs are used to control vomiting once an etiologic diagnosis is made, to prevent motion sickness and psychogenic vomiting and to control emesis from radiation or chemotherapy see The Merck Veterinary Manual, 8^{th} Ed, 1998, p 1682).

Apomorphine is a potent emetic (i.e induces vomiting).

Apomorphine-induced emesis model is a well-established canine model to investigate antiemetic actions of new compounds and has been used in studies investigating new NK1 antagonists (Furukawa et al. Biol Pharm Bull 36:974-979, 2013) as well as Cerenia and other antiemetic drugs used in dogs (Sedlacek et al. J Vet Pharmacol Therap 31:533-537, 2008).

In an embodiment, the dose of telmapitant is between about 1 mg/kg to about 10 mg/kg, or about 2 mg/kg to about 3 mg/kg or about 5 mg/kg to about 7 mg/kg or about 5 mg/kg or about 7 mg/kg.

In an embodiment the % w/v of telmapitant is between about 1% and about 10% or between about 2% and about 9% or between about 3% and about 8% or between about 5% and about 7% or about 5% or about 7%.

In an embodiment, the non-aqueous solvent is an oil.

In an embodiment, the non-aqueous solvent is oleic acid.

In another embodiment, the non-aqueous solvent is a fatty acid, a long chain triglyceride, a medium chain triglyceride or mixtures thereof.

In an embodiment, the non-aqueous solvent is Miglyol 810 or Miglyol 812N or mixtures of both.

In an embodiment, the composition comprises one or more surfactants.

In an embodiment, the surfactant is d-α-tocopherol polyethylene glycol 1000 succinate (TPGS), phosphatidylcholine or mixtures thereof.

In an embodiment, the surfactant is a polyethoxylated castor oil, such as Cremophor RH 40, Cremophor RH 60, Cremophor EL, Polyoxyl 15 Hydroxystearate (Solutol HS 15®), polyethoxylated sorbitan fatty acid esters such as polysorbate 20, 40, 60, or 80, polyoxyethylene-polyoxypropylene block copolymers such as poloxamer 188, 181, or 407 and sucrose fatty acid esters or mixtures thereof.

In an embodiment, the composition comprises an additional solvent.

In an embodiment, the additional solvent is propylene glycol.

In an embodiment, the additional solvent is 2-pyrrolidone.

In an embodiment, the additional solvent is diethylene glycol monoethyl ether

In an embodiment, the composition comprises an antioxidant.

In an embodiment, the antioxidant is ethylenediaminetetraacetic acid (EDTA) tetrasodium salt dihydrate.

In an embodiment, the antioxidant is EDTA, EDTA disodium, tocopherol, sodium metabisulfite, propyl gallate, ascorbic acid, ascorbyl palmitate, BHT, BHA, monothioglycerol or their combinations.

In an embodiment, the composition comprises a palatability agent.

In an embodiment, the palatability agent is sucralose, honey flavor or mixtures thereof.

An embodiment of the invention is an oral pharmaceutical composition comprising
a. telmapitant;
b. a mixture of TPGS and phosphatidylcholine;
c. propylene glycol; and
d. oleic acid;
wherein the telmapitant is in solution in the composition.

An embodiment of the invention is an oral pharmaceutical composition comprising
a. telmapitant;
b. a mixture of TPGS and phosphatidylcholine;
c. 2-pyrrolidone;
d. ethanol; and
e. Caprylic capric triglyceride;
wherein the telmapitant is in solution in the composition.

An embodiment of the invention is an oral pharmaceutical composition comprising
a. telmapitant;
b. diethylene glycol monoethyl ether; and
c. oleic acid;
wherein the telmapitant is in solution in the composition.

In an embodiment, the composition further comprises ethylenediaminetetraacetic acid tetrasodium salt dihydrate.

In an embodiment, the composition further comprises sucralose, honey flavor or mixtures thereof.

In an embodiment, the administration is of a single dose of the oral pharmaceutical composition.

In an embodiment, the anti-emetic effects last for at least 7 days.

In an embodiment, the animal is a mammal or a bird.

In an embodiment, the animal is a dog.

In an embodiment, the animal is a cat.

In an embodiment, the animal is a parakeet or a parrot.

In an embodiment, the animal receives radiation therapy or chemotherapy simultaneously or sequentially to the administration of the oral pharmaceutical composition.

In an embodiment, the animal shows emesis due to gastrointestinal disorders.

In an embodiment, the animal shows emesis due to motion sickness.

The claimed compositions are administered alone or in combination with food or drink water.

### EXAMPLES

### Example 1-1: Comparative Formulations

### Comparative Formulation 1—Injectable Formulation

| Component | % w/v |
|---|---|
| Telmapitant | 2.5 |
| Soluplus | 4 |
| TPGS | 4.18 |
| Ethanol | 7.2 |
| Water | QS |
| lactic acid | 2.5 |

This composition was formulated to deliver a dose of 5 mg/kg to the animals.

### Comparative Formulation 2—Compressed Tablets were Prepared with the Following Composition by a Wet Granulation Process

Tablet

| Component | % w/w |
|---|---|
| Telmapitant | 35.25 |
| PVP K30 | 1.9 |
| Poloxamer 188 | 0.48 |
| Liver flavor | 17.1 |
| Crospovidone | 9.75 |
| Avicel PH102 | 23.75 |
| Lactose monohydrate | 13.0 |
| Mg stearate | 0.76 |

### Comparative Formulation 3—a Liquid Suspension Formulation was Prepared with the Following Composition

Aqueous Suspension

| Component | % w/v |
|---|---|
| Telmapitant | 7 |
| Xanthan gum | 0.25 |
| Poloxamer 188 | 0.5 |
| Propylene glycol | 5.0 |
| Methylparaben | 0.2 |
| Propylparaben | 0.02 |
| Water | QS |
| Simethicone | 0.4 |
| Sucralose | 0.2 |
| Honey flavor | 1 |

### Example 1—2: Non-Aqueous Solution Formulations—Formulations According to the Invention

Formulation 1A

| | Composition | |
|---|---|---|
| Component | (% w/v) | Role |
| Telmapitant | 5.0-7.0 | Active |
| | | ingredient |
| d-α-Tocopherol polyethylene | 20.0 | surfactant |
| glycol 1000 succinate (TPGS) | | |
| Phosal ® 50 PG (liquid) | 10.0 | surfactant |
| Propylene glycol (liquid) | 30.0 | Co-solvent |
| Oleic acid (liquid) | QS | Non-aqueous |
| | | solvent |
| Ethylenediaminetetraacetic acid | 0.01 | Anti-oxidant |
| tetrasodium salt dihydrate (solid) | | |
| Sucralose (solid) | 0.5 | Palatability |
| | | agent |
| Honey flavor (liquid) | 1.0 | Palatability |
| | | agent |

Manufacturing Process
1. Melted TPGS
2. Combined liquid excipients; mixed with heat until a uniform clear solution achieved.
3. Added solid excipients and mixed to disperse
4. Added telmapitant and mixed while heating until fully dissolved.

Formulations 1B & 1C

| | 1B | 1C |
|---|---|---|
| Component | % w/v | % w/v |
| Telmapitant | 7 | 7 |
| TPGS | 5 | — |
| Phosal 50 PG | 60 | — |
| Propylene glycol | — | — |
| Oleic acid | — | QS |
| 2-pyrrolidone | 2.5 | — |
| Ethanol | 10 | — |
| Miglyol ® 812N | QS | — |
| Transcutol HP | — | 35 |
| Sucralose | 0.5 | 0.5 |
| Honey flavor | 2 | 1 |
| EDTA 4Na | 0.01 | 0.01 |

Formulations 1B and 1C were made by processes similar to Formulation 1A above.

### Example 2

Comparative Example Formulations 1, 2 and 3 and Formulations 1A, 1B and 1C were evaluated in pharmacokinetic studies. Study design: Mature healthy Beagle dogs (group size: 5-6 dogs) were used for the PK studies. After single administration of telmapitant, blood collection for analysis of plasma concentrations was performed at different time points for up to 336 hours after administration. The concentration of telmapitant in Formulation 1A was adjusted to 7% w/v for comparison with the other liquid formulations which have a telmapitant concentration of 7% w/v.

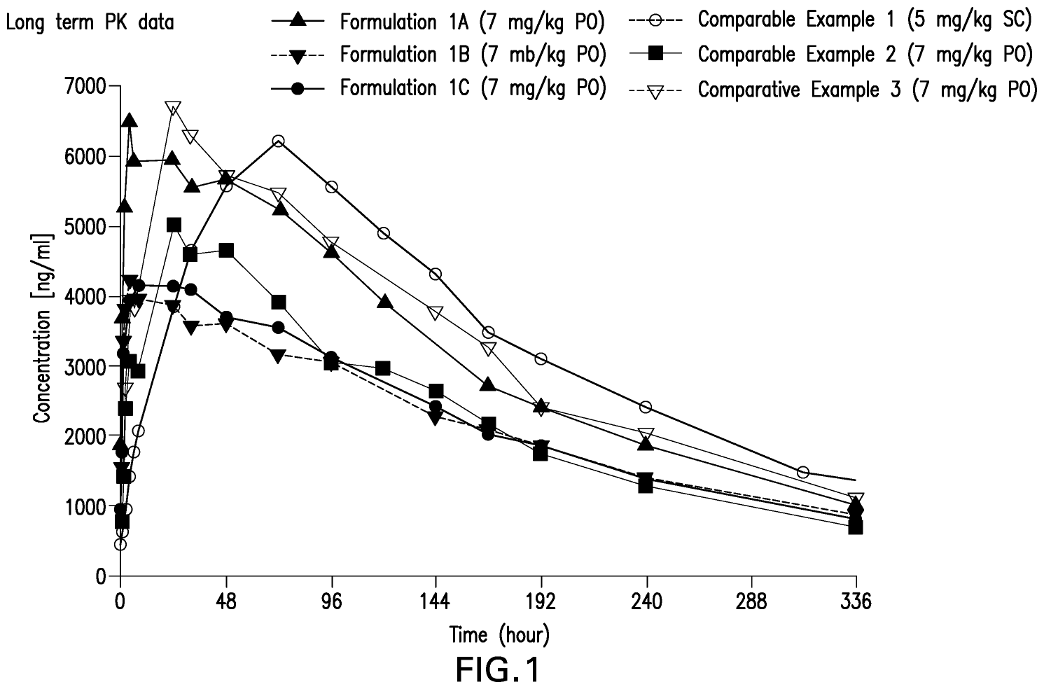

The results are presented in FIGS. 1-3A-3E. Formulations according to the invention have blood levels indicative of a rapid onset of activity; have low inter-animal variability and superior maximum concentrations of telmapitant in the blood.

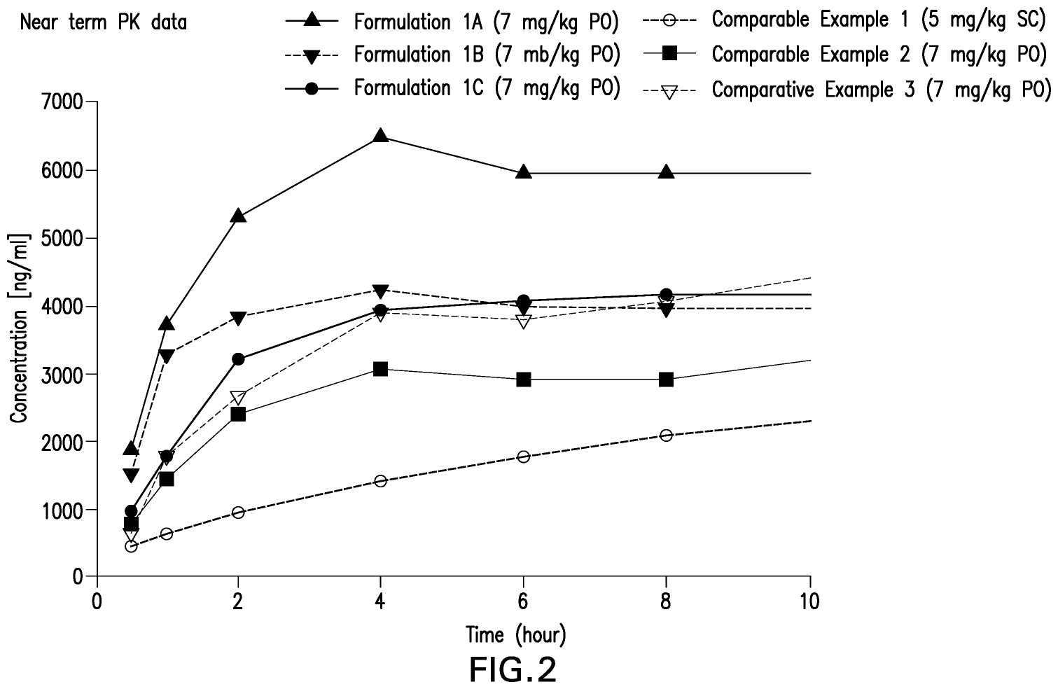

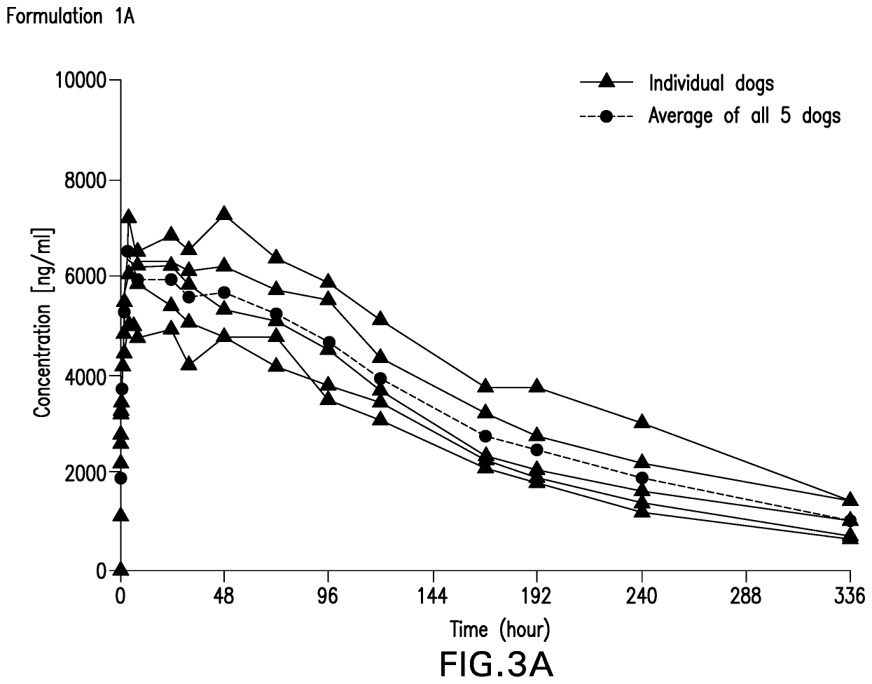

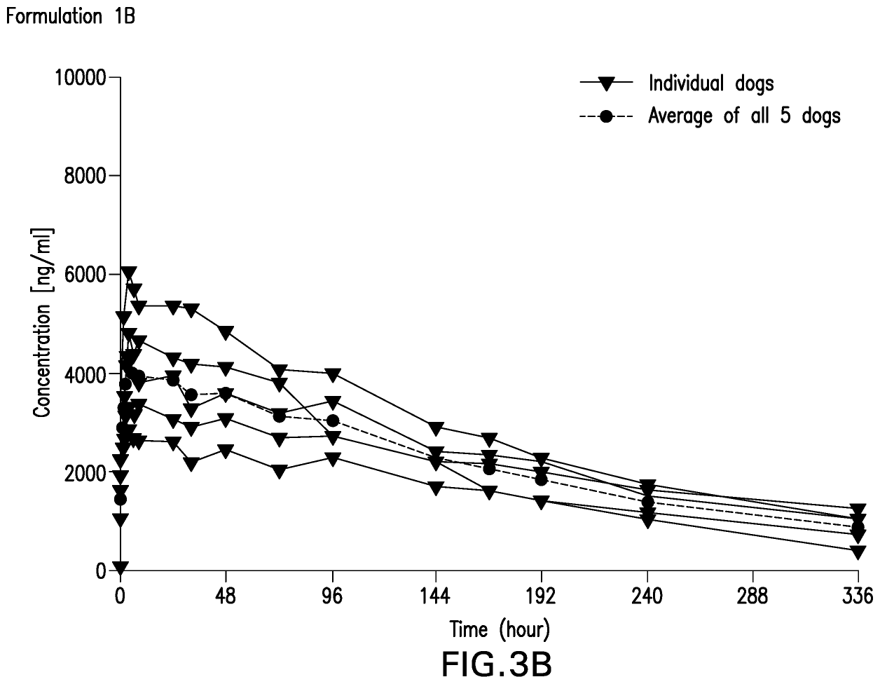

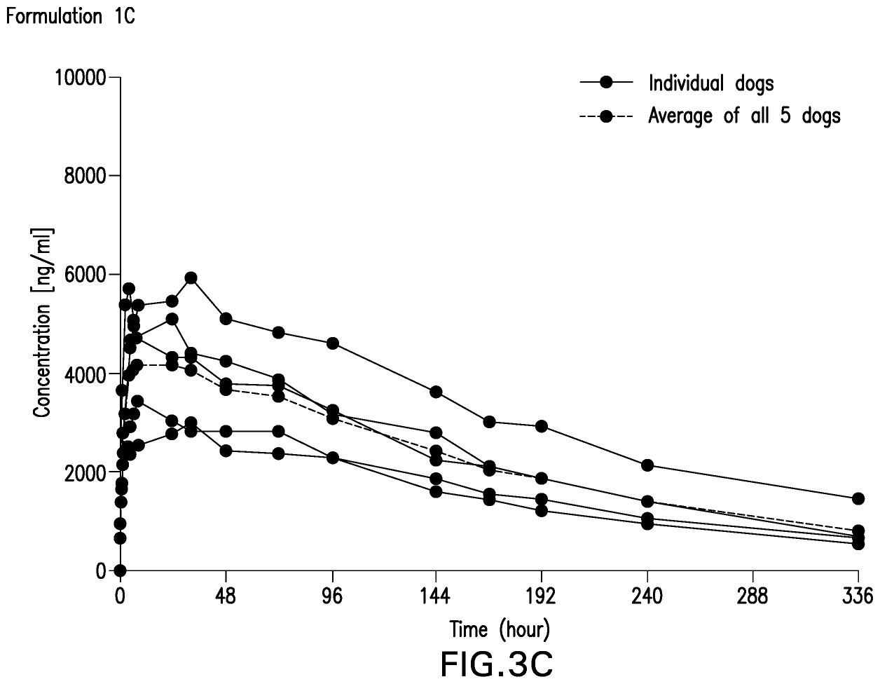

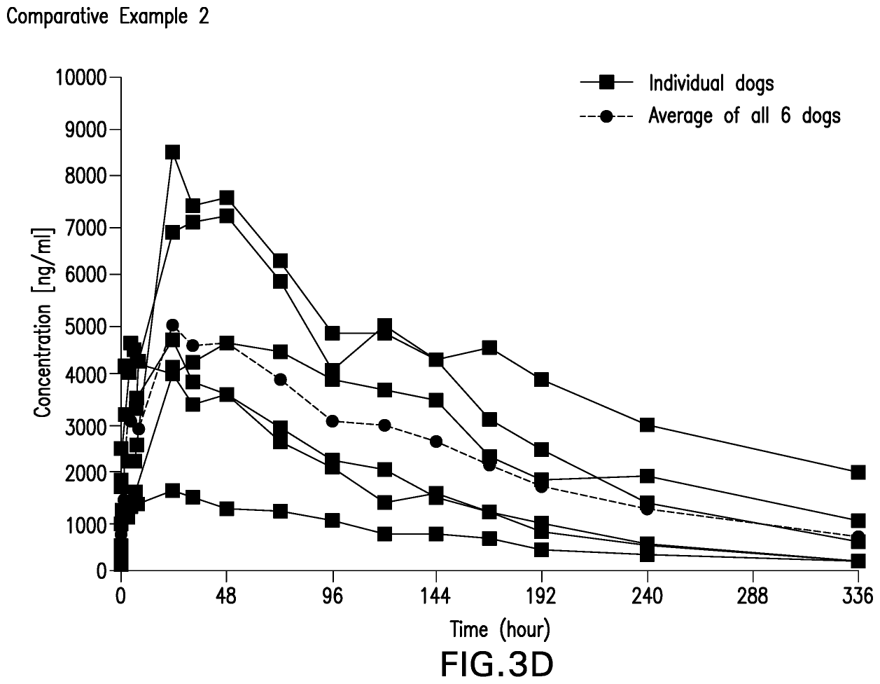

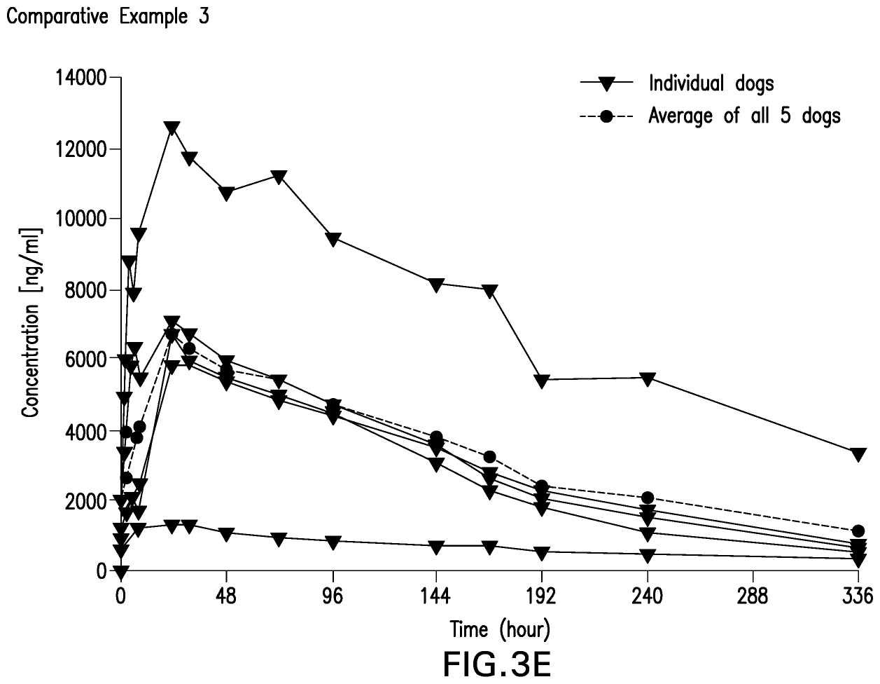

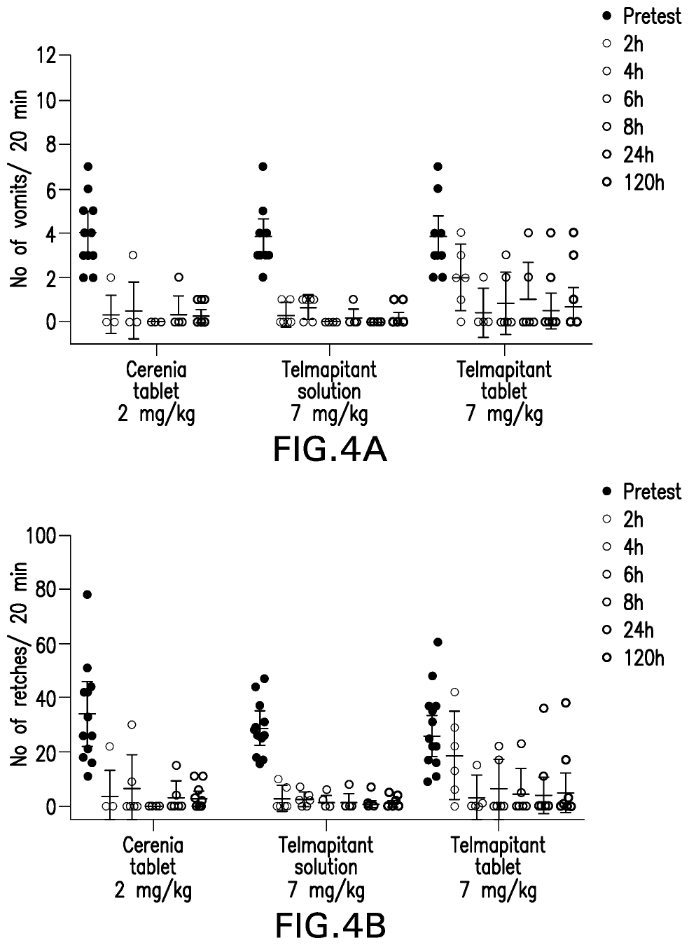

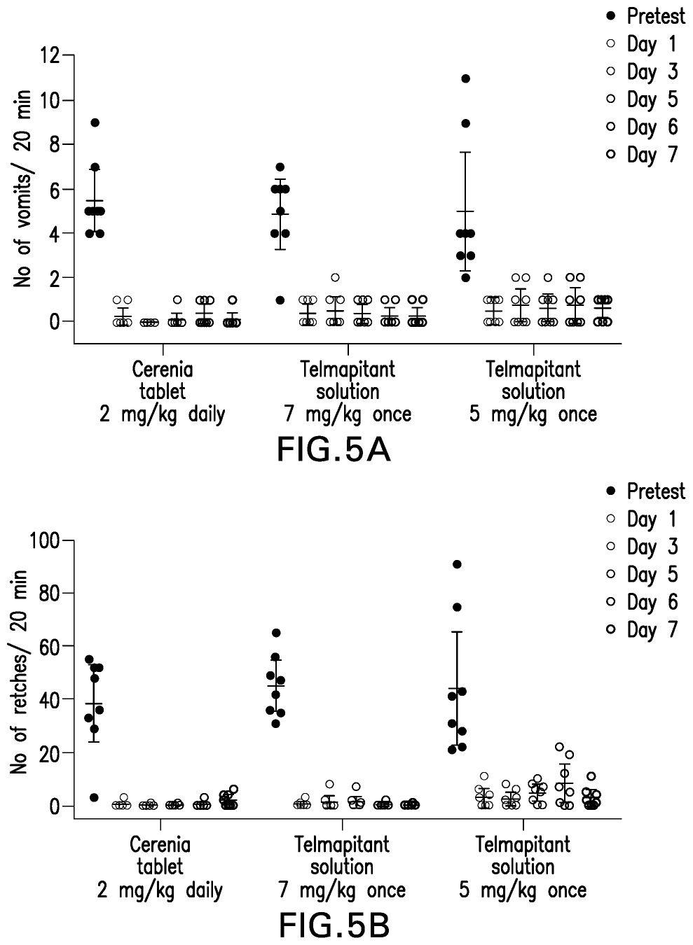

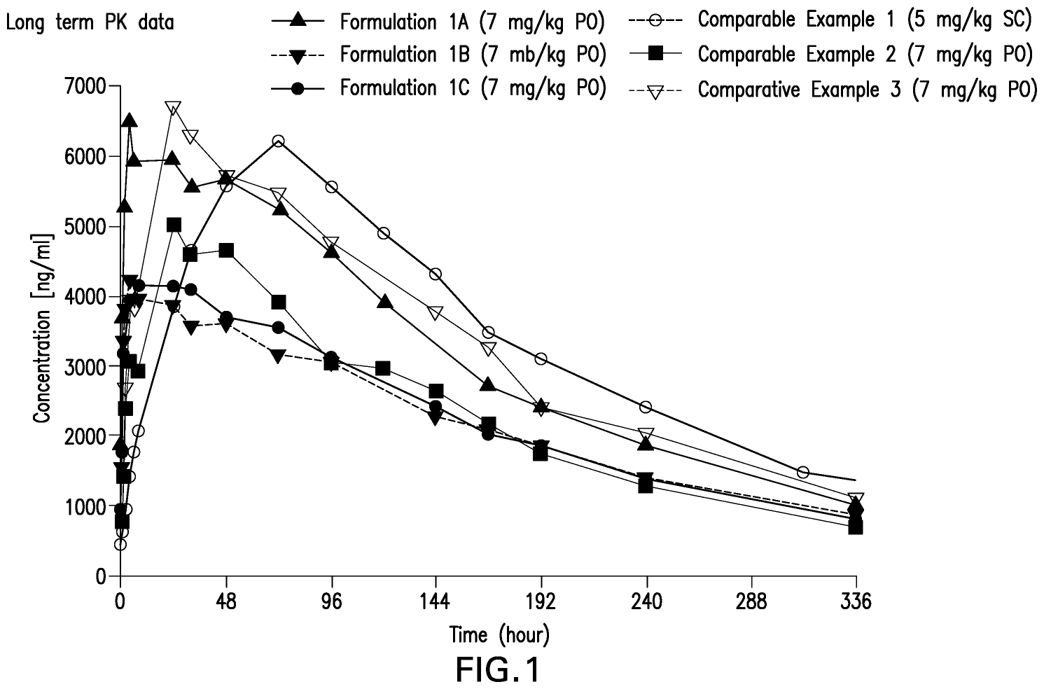

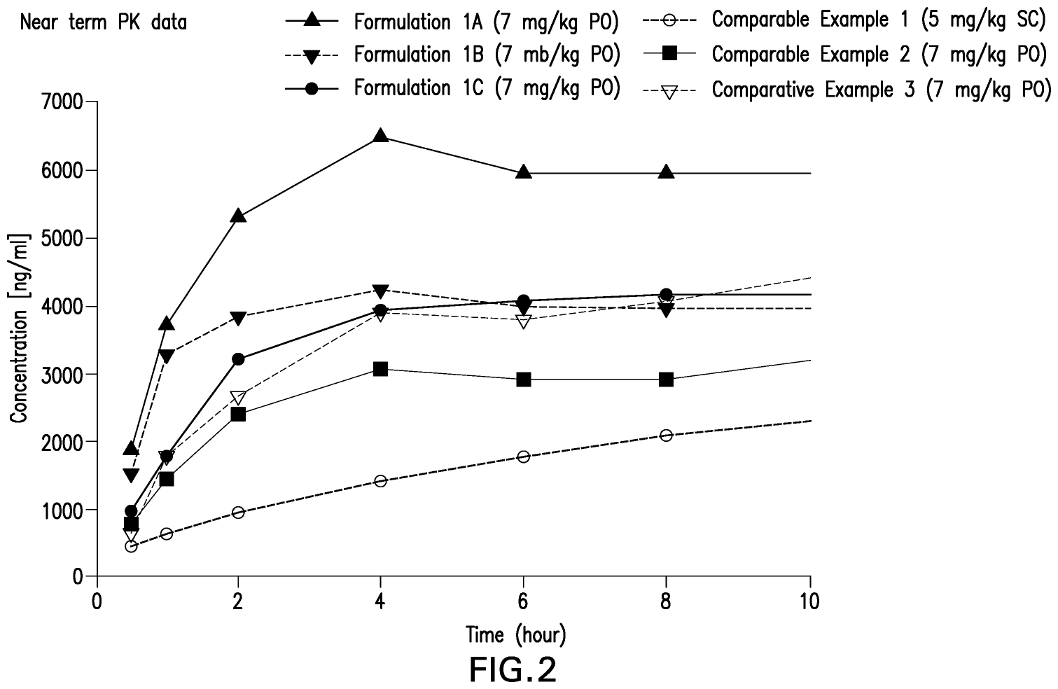

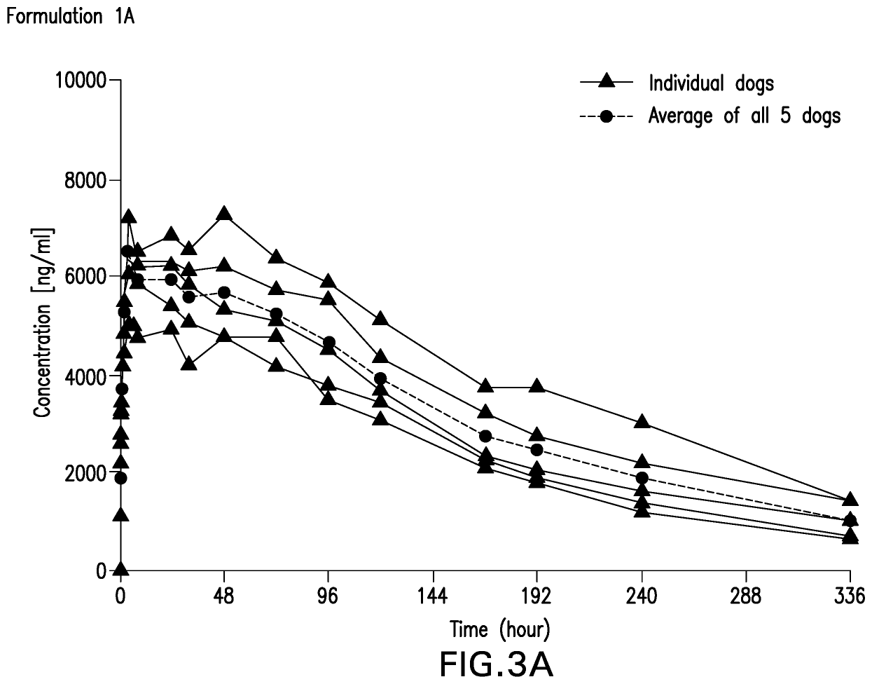

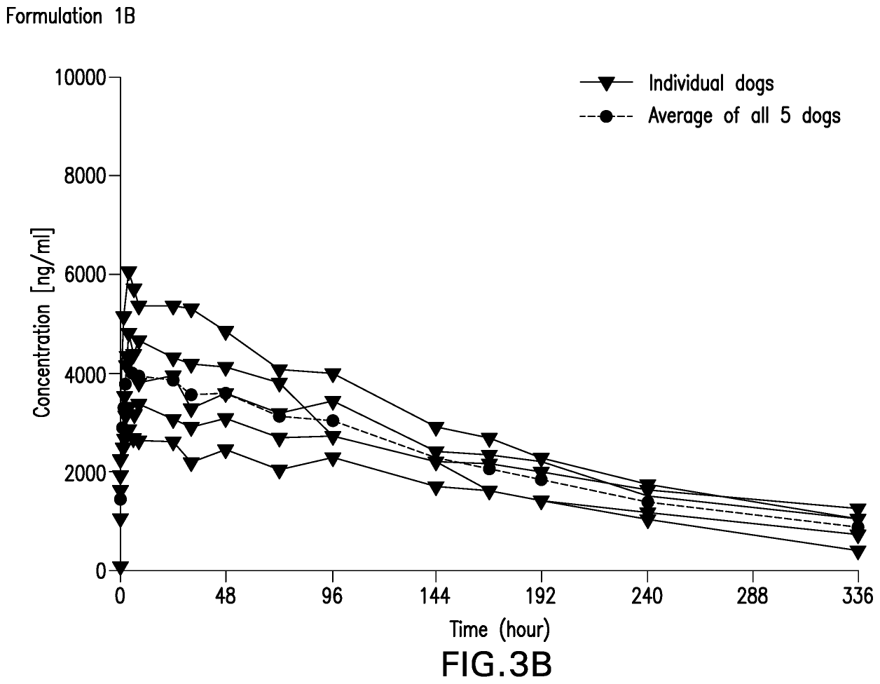

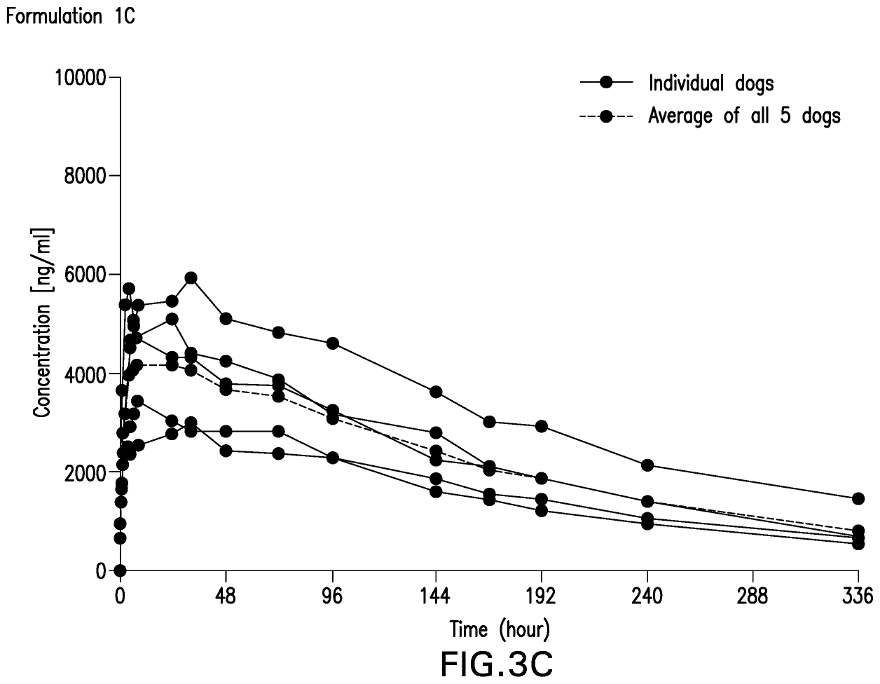

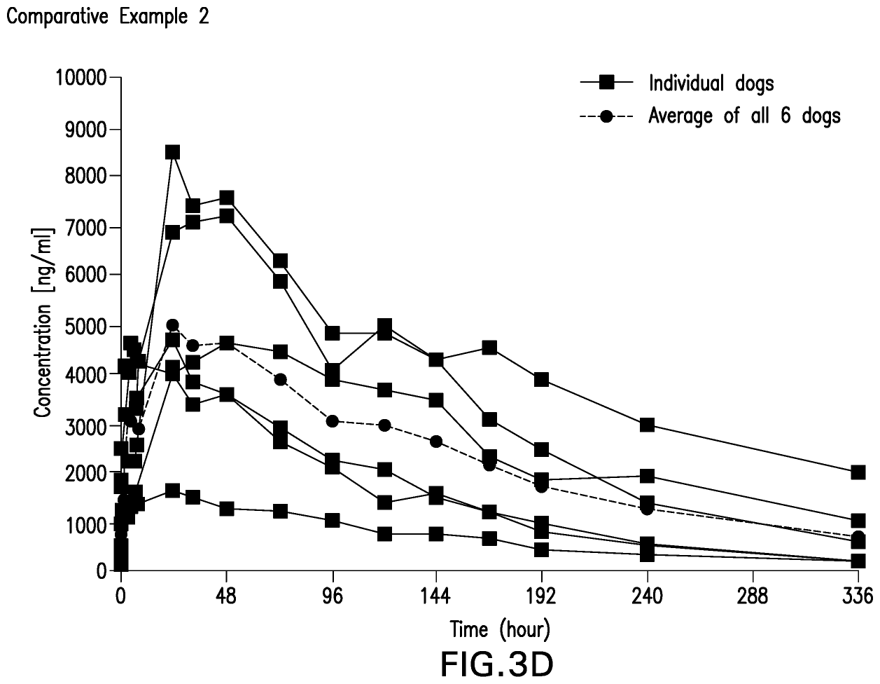

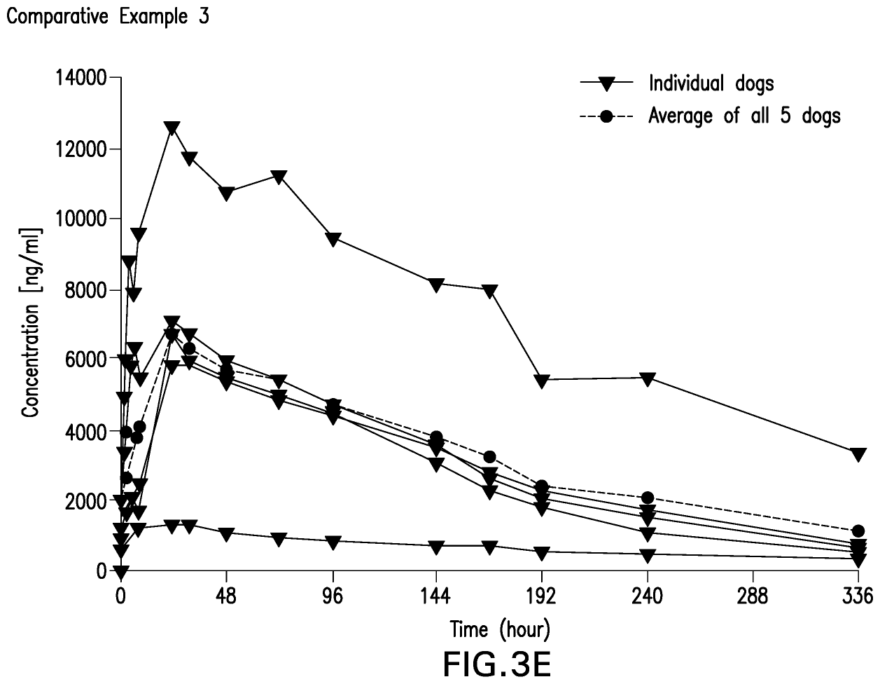

FIG. 1 shows that Formulation 1A, and Comparative Example Formulations 1 and 3 have superior Cₘₐₓ levels compared to the other formulations. FIG. 2 shows Formulation 1A demonstrated superior (shorter) Tₘₐₓ than any of the other formulations. FIGS. 3A-3E display the variability in PK profiles exhibited between different animals tested with each formulation. Comparative Example Formulations 2 (tablet) (FIG. 3D) and 3 (aqueous suspension) (FIG. 3E) exhibited great inter animal variability whereas the solution formulations displayed comparatively less inter animal variability. Formulation 1A (FIG. 3A) showed the least interanimal variability.

Table 1 also shows the interanimal variability of the above mentioned PK studies.

TABLE 1

| Formulation | % CV, T ½ | % CV, Cₘₐₓ | % CV, AUC_{inf} |
|---|---|---|---|
| 1A (Liquid, non- | 26.7 | 21.2 | 28.8 |
| aqueous) | | | |
| 1B (Liquid, non- | 36.5 | 29.4 | 29.6 |
| aqueous) | | | |
| Comparative | 35.0 | 48.3 | 56.3 |
| Formulation 3 | | | |
| (Liquid, aqueous) | | | |
| Comparative | 40.4 | 44.3 | 60.3 |
| Formulation 2 | | | |
| (Tablet) | | | |
| Comparative | 35.9 | 23.0 | 43.6 |
| Formulation 1 | | | |
| (subcutaneous | | | |
| injection) | | | |

Coefficient of Variation (CV) for three PK parameters in percent after administration of different formulations via oral and subcutaneous routes. Average values of three to six separate experiments are shown. Half-life (T1/2), maximum plasma concentration (Cₘₐₓ), and the area under the curve calculated to infinity (AUC_{inf}) were selected as measures for drug exposure and duration of drug presence in the body. The lowest interanimal variability for all three parameters was observed with Formulation 1A.

### Example 3—Apomorphine-Induced Emesis Model

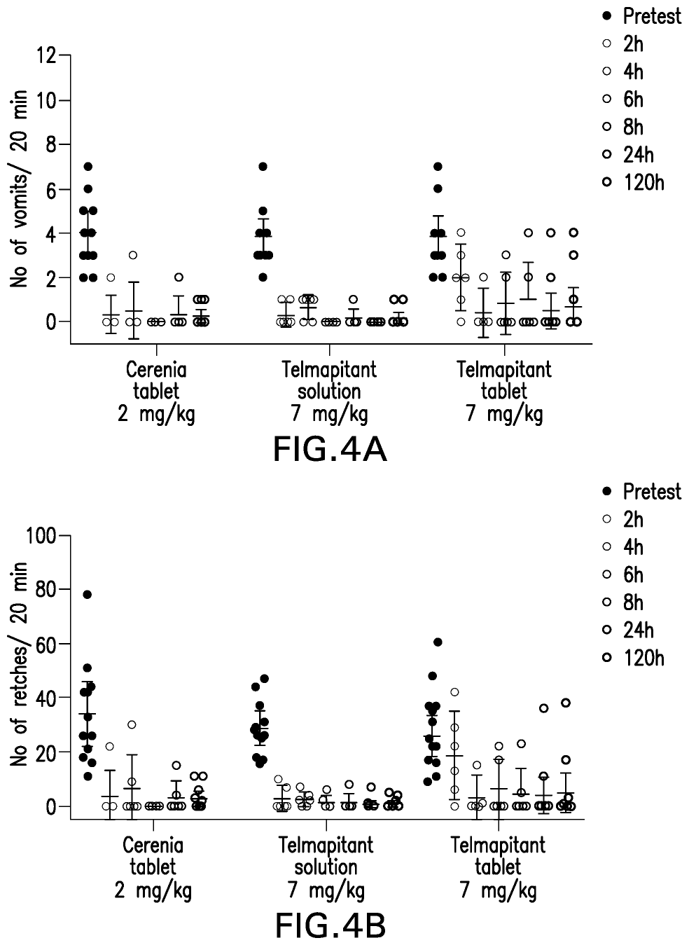

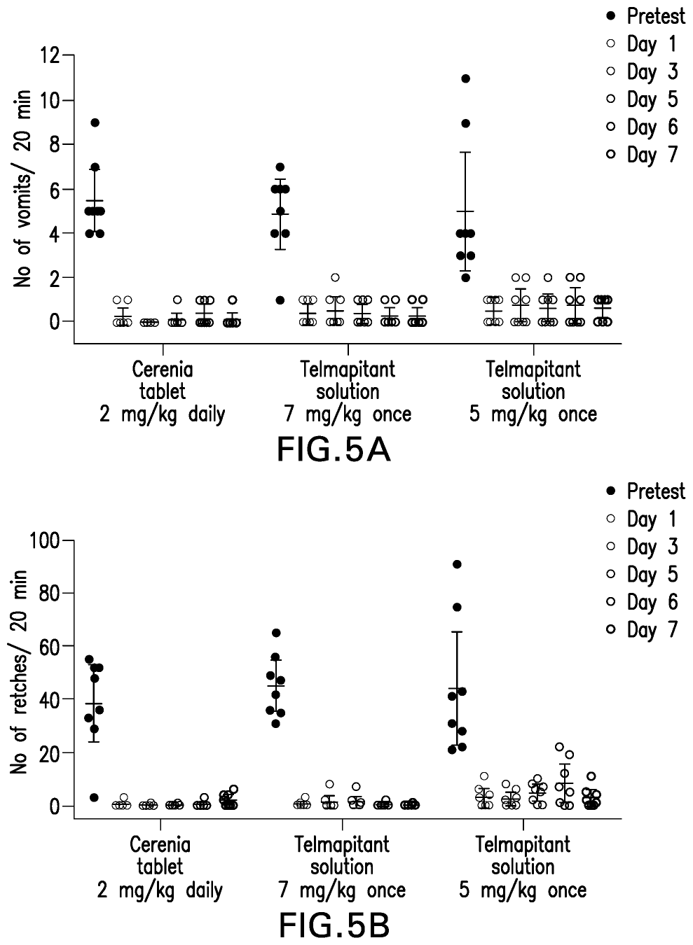

The canine model of apomorphine-induced emesis is a well-established model to investigate antiemetic actions of new compounds and has been used in studies investigating new NK1 antagonists (Furukawa et al. Biol Pharm Bull 36:974-979, 2013) as well as Cerenia and other antiemetic drugs used in dogs (Sedlacek et al. J Vet Pharmacol Therap 31:533-537, 2008). This model was used to evaluate the antiemetic activity of telmapitant in non-aqueous solution formulations and solid formulations (FIGS. 4A and 4B). The onset of action (FIGS. 4A and 4B) as well as the duration of activity (FIGS. 5A and 5B) were investigated after a single oral administration. Intravenous (IV) administration of apomorphine (emetogenic challenge 0.03 mg/kg) was delivered to all individuals 2 h, 4 h, 6 h, 8 h, 24 h, and 120 h (FIGS. 4A and 4B) or 1 d, 3 d, 5 d, 6 d, and 7 d (FIGS. 5A and 5B). Dogs were dosed orally with a single administration at 5 or 7 mg/kg body weight (BW) of telmapitant solution on Day 0 or daily doses of Cerenia® tablets at 2 mg/kg BW. Cerenia® (maropitant) at recommended dose and daily administrations was used as a positive control. Any emetic events (vomiting episodes and/or retches) following each emetogenic challenge were recorded for 20 min. In pre-test periods, all the dogs responded to the emetogenic challenge, similarly in all groups. Moreover, the response remained stable for the duration of the studies (120 h or 7 days, respectively).

FIGS. 4A and 4B show that the telmapitant non-aqueous solution formulation (7% telmapitant, no EDTA added) provides a rapid onset of antiemetic action comparable to the positive control. The solid tablet formulation appeared to be inferior regarding onset of action and antiemetic effects. Cerenia® (maropitant) was administered at 2 mg/kg and telmapitant at 7 mg/kg. The dogs were challenged with intravenous apomorphine pre-dose (pretest) and at 2, 4, 6, 8, 24, and 120 hours (telmapitant only) post dose. The shown data are from 6-12 animals per group. Bars show average and standard deviation (SD).

A single oral dose of telmapitant non-aqueous solution formulation revealed antiemetic effects for at least 7 days in the apomorphine-induced emesis model in the dog (FIGS. 5A and 5B). Findings from two different experiments were combined in the graphs of FIGS. 5A and 5B. The anti-emetic efficacy of a single administration of telmapitant non-aqueous solution formulation at 7 mg/kg was comparable to the antiemetic response of daily administrations of Cerenia®. Data of telmapitant at 5 mg/kg were not significantly different from 7 mg/kg. Cerenia® (maropitant) was administered in daily doses of 2 mg/kg and telmapitant in single doses of 5 and 7 mg/kg. Dogs were dosed orally with a single dose of 5 or 7 mg/kg body weight (BW) of telmapitant on Day 0 or daily doses of Cerenia® at 2 mg/kg BW. The dogs were challenged with intravenous apomorphine pre-dose (pretest) and at 1, 3, 5, 6, and 7 days post dose. The shown data are from 8 animals per group. Bars show average and SD.

### Example 4—Palatability Study

| | Formulation 1A | | Formulation 1B | |
|---|---|---|---|---|
| Breed | Active | Placebo | Active | Placebo |
| Beagle | (16/20) 80% | (19/20) 95% | (11/20) 55% | (14/20) 70% |
| Labrador | (10/10) 100% | | (8/10) 80% | |
| Cross- | (9/10) 90% | | (7/9) 78% | |
| breed | | | | |

Formulations administered orally to dogs, 2 period cross-over design. After each individual treatment, the acceptance of the formulation was established by two different observers using a scoring system (0-3). The percentages of dogs that accepted (score ≤1) and did not accept (score ≥2) were calculated. A formulation was qualified as “accepted” if at least 70% of dogs accepted it and as “not-accepted” if more than 30% of dogs did not accept it.

Formulation 1A was more palatable to all of the tested species of dogs than Formulation 1B.

1. An oral pharmaceutical composition comprising telmapitant, a non-aqueous solvent and one or more additional pharmaceutical acceptable excipients wherein the telmapitant is in solution in the composition.

2. The composition of claim 1, wherein the non-aqueous solvent is oleic acid.

3. The composition of claim 1, wherein the composition comprises one or more surfactants.

4. The composition of claim 3, wherein the surfactant is d-α-tocopherol polyethylene glycol 1000 succinate (TPGS), phosphatidylcholine or mixtures thereof.

5. The composition of claim 1, wherein the composition comprises an additional solvent.

6. The composition of claim 5, wherein the additional solvent is propylene glycol.

7. The composition of claim 1, wherein the composition comprises an antioxidant.

8. The composition of claim 7, wherein the antioxidant is ethylenediaminetetraacetic acid tetrasodium salt dihydrate.

9. The composition of claim 1, wherein the composition comprises a palatability agent.

10. The composition of claim 9, wherein the palatability agent is sucralose, honey flavor or mixtures thereof.

11. An oral pharmaceutical composition comprising
a. telmapitant;
b. a mixture of TPGS and phosphatidylcholine;
c. propylene glycol; and
d. oleic acid;
wherein the telmapitant is in solution in the composition.

12. The oral pharmaceutical composition of claim 11, further comprising ethylenediaminetetraacetic acid tetrasodium salt dihydrate.

13. The oral pharmaceutical composition of claim 11, further comprising sucralose, honey flavor or mixtures thereof.

14. A method of treating or preventing emesis in an animal comprising administering orally to the animal an effective dose of the oral pharmaceutical composition of claim 1.

15. The method of claim 14, wherein the administration is of a single dose of the oral pharmaceutical composition.

16. The method of claim 14, wherein the anti-emetic effects last for at least 7 days.

17. The method of claim 14, wherein the animal is a dog.

18. The method of claim 14, wherein the animal is a cat.

19. The method of claim 14, wherein the animal receives chemotherapy simultaneously or sequentially to the administration of the oral pharmaceutical composition.

20. The oral pharmaceutical composition of claim 12, further comprising sucralose, honey flavor or mixtures thereof.
